(19) United States
(12) Patent Application Publication
(10) Pub. No.: US 2003/0191505 A1
(43) Pub. Date: Oct. 9, 2003

(54) Magnetic structure for feedthrough filter assembly

(76) Inventors: Mark Gryzwa, Woodbury, MN (US); Allen Novotny, Zimmerman, MN (US); David Chizek, Fridley, MN (US); Jason Sprain, Shoreview, MN (US); Michael J. Lyden, Shoreview, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/119,543

(22) Filed: Apr. 9, 2002

(51) Int. Cl. A61N 1/372

(52) U.S. Cl. 607/37

(57) ABSTRACT

A feedthrough assembly for use in an implantable medical device that performs filtering of electromagnetic interference and can be easily manufactured. A magnetic structure is adapted to fit over a plurality of terminal pins of the feedthrough assembly within the device housing to provide inductive isolation from electromagnetic interference.

### FIELD OF THE INVENTION

This invention pertains to cardiac rhythm management devices such as pacemakers, implantable cardioverter/defibrillators, and implantable monitoring devices.

### BACKGROUND

Implantable medical devices such as pacemakers and implantable cardioverter/defibrillators include electronic circuitry that is enclosed within a housing made of biocompatible material such as titanium that protects the circuitry from body fluids. These devices also utilize external lead wires that conduct signals from sensing electrodes to the electronic circuitry within the housing. Some means must therefore be provided that permits the passage of the lead wires, or other conductors to which the lead wires are connected, through the wall of the housing while maintaining a hermetic seal to prevent the entry of body fluids. Since the housing is made of conductive material, the conductors passing through the housing wall must also be insulated from the wall and from one another. The structure that provides this function is commonly referred to in the industry as a feedthrough assembly.

Electromagnetic interference from various external sources can adversely affect the operation of an implantable medical device if such interference is mixed with the sensing signals carried by the lead wires. The conductive housing of the device effectively shields the electronic circuitry from such interference, but the conductive lead wires are external to the housing. The lead wires can thus act as antennas for the interference so that the signals carried by the lead wires include undesired noise. A common way of dealing with this problem is for the feedthrough assembly to interpose some capacitance between the lead wires and the conductive housing. The feedthrough assembly then acts as a low-pass filter to effectively short the relatively high frequency electromagnetic interference to the conductive housing and remove it from the signal received by the electronic circuitry.

### SUMMARY

The present invention relates to a feedthrough filter assembly for an implantable medical device that provides both desirable filtering of electromagnetic interference and ease of manufacture. The assembly may include a conductive ferrule through which a plurality of conductive pins pass in non-conductive and sealing relation where the ferrule is adapted for fitting within an opening of a conductive housing. The assembly further includes a magnetic structure for fitting over the terminal pins inside the housing to provide inductive filtering and attenuation of noise due to electromagnetic interference.

### BRIEF DESCRIPTION OF THE DRAWINGS

FIG. 1 is a view of the bottom half of a conductive housing for an implantable medical device showing the interior thereof.

FIG. 2 shows an exemplary feedthrough filter assembly.

FIGS. 3A and 3B show alternate embodiments of a magnetic structure for incorporating into the feedthrough assembly.

### DETAILED DESCRIPTION

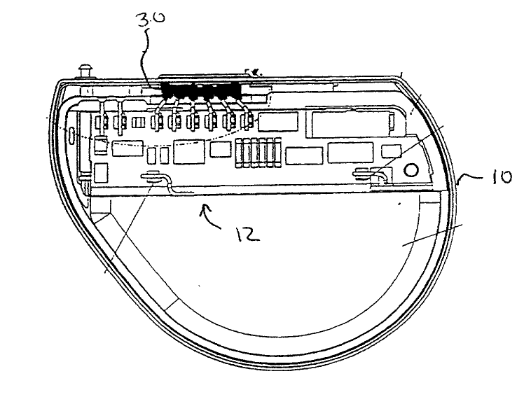

FIG. 1 is a depiction of an exemplary implantable medical device in which may be incorporated the present invention. The device may be a cardiac rhythm management device, such as a pacemaker or implantable cardioverter/defibrillator, that senses intrinsic cardiac activity and delivers electrical stimulation to the heart. Shown in FIG. 1 is a housing 10 that encloses the internal circuitry 12 used for processing sensing signals and delivering electrical stimulation in the form of pacing pulses or defibrillation shocks. The housing may be constructed of two portions, one of which is shown in FIG. 1, that are sealed together during final assembly and is designed to be implanted subcutaneously on a patient's chest. Lead wires from the housing can then be threaded intravenously into the heart to connect the device to electrodes used for sensing electrical activity and delivery of electrical stimulation. The housing 10 is a sealed container that protects the internal circuitry from body fluids and is constructed of a biocompatible material such as titanium that also shields the internal circuitry from electromagnetic interference.

As aforesaid, a feedthrough assembly is a structure that allows signal conductors connected to the lead wires to enter the housing 10 and connect to the internal circuitry 12 in a manner that maintains a fluid-tight seal. FIG. 2 shows a feedthrough assembly that includes a ferrule 20 and a plurality of terminal pins 22 that pass from one side of the ferrule to the other. The ferrule is constructed of titanium or other biocompatible metal and is adapted to sealingly fit within an opening in the wall of the housing 10 so that one side of the ferrule faces the interior of the housing and the other side faces toward the exterior. The end of a terminal pin external to the housing connects to a lead wire, while the end internal to the housing connects to the internal circuitry. The terminal pins are sealingly inserted through the ferrule in nonconductive relation. FIG. 2 shows an embodiment in which the terminal pins 22 pass through insulating bushings 24 that are mounted within the ferrule and form a fluid-tight seal.

After the device is implanted, the intravenously placed lead wires are external to the conductive housing and can pick up electromagnetic interference. To deal with this problem, a low-pass filter can be placed in the signal path to attenuate the relatively high-frequency electromagnetic interference while still allowing transmission of cardiac signals and delivery of stimulation pulses through the lead wires. One way of implementing such low-pass filtering is to interpose capacitance between the terminal pins and the conductive ferrule in the feedthrough assembly, where the ferrule and housing are used as a signal ground. For example, the bushings 24 in FIG. 2 may incorporate a structure with material of an appropriate dielectric constant so that high frequencies are shorted to the conductive ferrule. Many other different types of capacitive structures can be utilized in a feedthrough assembly to provide this filtering function.

Isolation from the effects of electromagnetic interference can also be brought about by adding inductance to the signal path between the terminal pins and the internal circuitry. Inductance can be added by surrounding a portion of the signal conductor with a magnetic structure made of, for example, a ferrimagnetic material such as ferrite. One way to do this is to incorporate ferrite beads around each terminal pin within the conductive ferrule. An easier to manufacture method, however, is to use a magnetic structure that is fit over a plurality of terminal pins or other signal conductors on the side of the conductive ferrule within the device housing. Unlike as would be the case with a capacitor, adding inductance in this manner does not require that an electrical connection be established with the signal conductor by soldering or with conductive epoxy which would incrementally add to manufacturing costs. FIGS. 1 and 2 show such a magnetic structure 30 fitted over a plurality of the terminal pins 24.

FIG. 3A shows such a magnetic structure 30 that is fitted over a plurality of signal conductors by inserting the conductors through a plurality of holes. FIG. 3B shows an alternative embodiment in which the magnetic structure comprises two half-portions 30a and 30b that are fit over the signal conductors and attached together. The surface of the magnetic structure 30 is made non-conductive in order to maintain electrical isolation of the signal conductors from one another. In the case of ferrite and most other ferrimagnetic materials, the surface of the structure 30 is oxidized by natural means or otherwise to form a non-conducting surface.

The magnetic structure 30 can be used either alone or in conjunction with capacitance located in the feedthrough assembly or elsewhere to perform the low-pass filtering of the signals conducted by the lead wires. The amount of inductance that needs to be added in order to result in a desired cut-off frequency depends upon the amount of capacitance in the circuit. An advantage with fitting the magnetic structure over the terminal pins within the device housing, as opposed to integrating it within the conductive ferrule, is that the size of the magnetic structure and amount of added inductance can be easily changed in accordance with other design changes to the device that affect capacitance. For example, some internal circuitry designs utilize multi-layer circuit boards that add capacitance to the signal path. In certain cases, this added capacitance is enough so that adding capacitance within the conductive ferrule is not necessary to achieve effective isolation from electromagnetic interference. Also, in cases where it is desired to use ferrule incorporating capacitance regardless of any internal circuitry capacitance, the amount of added inductance can then be changed accordingly to result in optimum filtering characteristics.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

What is claimed is:

1. A feedthrough filter assembly, comprising:
a plurality of conductive terminal pins;
a conductive ferrule through which the terminal pins pass in non-conductive and sealing relation, the ferrule being adapted for fitting within an opening of a conductive housing; and,
a magnetic structure for fitting over the terminal pins within the conductive housing to thereby form an inductive filter for signals carried by the terminal pins.

2. The assembly of claim 1 wherein the magnetic structure is made from a ferrimagnetic material.

3. The assembly of claim 2 wherein the ferromagnetic material is ferrite.

4. The assembly of claim 1 wherein the magnetic structure is a block having openings therein through which the terminal pins pass.

5. The assembly of claim 1 wherein the magnetic structure has a non-conductive surface that insulates the terminal pins from one another.

6. The assembly of claim 1 wherein the magnetic structure is made from ferrimagnetic material that has a non-conductive oxide surface.

7. The assembly of claim 1 further comprising a non-conductive bushing within the conductive ferrule through which the terminal pins pass.

8. The assembly of claim 1 further comprising one or more capacitors within the conductive ferrule to form a capacitive filter for signals carried by the terminal pins.

9. The assembly of claim 1 wherein the magnetic structure is formed of two or more components that are fitted over the terminal pins and fastened together.

10. A method for constructing a feedthrough filter assembly, comprising:
passing a plurality of conductive terminal pins through a conductive ferrule in non-conductive and sealing relation;
fitting the conductive ferrule within an opening of a conductive housing for an implantable medical device; and,
fitting a magnetic structure over the terminal pins within the conductive housing to thereby form an inductive filter for signals carried by the terminal pins.

11. The method of claim 10 wherein the magnetic structure is made from a ferrimagnetic material.

12. The method of claim 11 wherein the ferromagnetic material is ferrite.

13. The method of claim 10 wherein the magnetic structure is a block having openings therein through which the terminal pins pass.

14. The method of claim 10 wherein the magnetic structure has a non-conductive surface that insulates the terminal pins from one another.

15. The method of claim 10 wherein the magnetic structure is made from ferrimagnetic material that has a non-conductive oxide surface.

16. The method of claim 10 further comprising integrating a non-conductive bushing within the conductive ferrule through which the terminal pins pass.

17. The method of claim 10 further comprising integrating one or more capacitors within the conductive ferrule to form a capacitive filter for signals carried by the terminal pins.

18. The method of claim 10 further comprising fitting two half-portions of the magnetic structure over the terminal pins and fastening them together.
